(19) United States
(12) Patent Application Publication
(10) Pub. No.: US 2025/0329449 A1
(43) Pub. Date: Oct. 23, 2025

(54) HEALTH MONITORING SYSTEM AND METHOD

(71) Applicant: Margaret WOODBURY, White Plains, NY (US)

(72) Inventor: Margaret WOODBURY, White Plains, NY (US)

(21) Appl. No.: 18/641,762

(22) Filed: Apr. 22, 2024

(51) Int. Cl. G16H 40/67; G16H 80/00

(52) U.S. Cl. CPC G16H 40/67; G16H 80/00

(57) ABSTRACT

A monitoring system can notify at least one contact if a monitored user does not respond appropriately to an automated alert presented on the user's device (e.g., smartphone or tablet) within a configurable amount of time. The monitoring method and system can alert the user's designated contact person (e.g., a registered “in case of emergency” (ICE) contact) (or a hierarchy of people) to check in with the monitored user or their household or residence. The method and system may provide additional pet health and safety benefits in households with pets that are cared for primarily by the monitored user.

### BACKGROUND OF THE INVENTION

### Field of the Invention

The present disclosure relates to a health monitoring system and method, and, in one embodiment, to a method and system for performing periodic wellness checks on persons living alone, or single parents with young children, or those temporarily alone such as traveling/hiking, spouse/partner away, or taking a new medication, using mobile devices (e.g., smartphones or tablets).

### Discussion of the Background

From 1960 to 2022, the number of single-person households in the US skyrocketed from 6.9 million to 37.9 million—an increase of 443 percent. There are now some 38 million single-person households in the United States. The median age of marriage has steadily increased from the 1960s—from a median age of ˜21 for women and ˜22 for men—to median ages of 28 and 30, respectively, in 2022. (By age 60, in the US, 27 percent of adults live alone.) And while coupled households remain the majority (53%), their dominance is rapidly shrinking. In 2000, 57 percent of households were coupled—i.e., householder has spouse or partner living with them. Added to this mix is an aging US population—in 2020, 65 and older people make up 17 percent of the US population, or 1 in 6 ppl; by way of comparison, in 1920 this demographic was only 1 in 20 of total population. (Among peer nations, the US lags behind many others in percentage of ppl age 65+, with Japan, Italy, Greece and Germany, topping the list—all with >20% of their total population >65.) Globally this trend towards single-person households is expanding rapidly as well.

Dovetailing this trend is a rise in obesity, overdose deaths and ill health. A September 2022 study out of Cedars Sinai (https://doi.org/10.1002/jmv.28187) found a 30 percent increase in heart attack deaths among youth age 25 to 44 years of age in recent years; a 20 percent increase in those aged 45 to 64; and a 14 percent increase in adults over age 65. Overdose deaths in the US have risen steadily since the late 1990s (less than 20,000 per annum) to a staggering 107,000 in 2021. Globally, the number of people who suffer from drug use disorders has increased 45 percent in the past ten years, per a 2023 report by the United Nations. Obesity rates (a driver of numerous serious health maladies) have grown exponentially as well over the past three decades: Globally, the obesity rate in adults is up ˜30 percent, and in children has risen by 47 percent in the same time frame. It has been projected that by middle age, ˜70% of millennials will be obese. In the US, 20% of children now present with obesity. And a CDC study has found a significant increase in the rate of young adult suicides over the past two decades.

As populations trend towards single-person households and greater levels of ill-health/lifestyles, there is an ever-growing need to have an alert system in place that will assure that a designated in-case-of-emergency person is notified if the human in a household becomes non-responsive. Moreover, some 70 percent of all US households have pets and worldwide there are approximately 500 million pet dogs and 400 million pet cats. Many other households have fish, turtles and other manner of non-human pets. However, should a pet owner (or the human responsible for the pet) living alone become incapacitated, deceased or otherwise cannot signal for help, the safety of the pet as well the person can become in jeopardy.

There exist systems that a conscious person can use to call for help. These are designed primarily to preserve the life/health of the client user. However, such systems are not of use if the monitored person is deceased or unconscious, or otherwise restricted due to crime victimization.

### SUMMARY OF EXEMPLARY EMBODIMENTS

There exists a need to have a convenient monitoring system that can notify at least one contact if a monitored user does not respond appropriately to an automated alert presented on the user's device (e.g., smartphone or tablet) within a configurable amount of time. The monitoring method and system can alert the user's designated contact person (e.g., a registered “in case of emergency” (ICE) contact) to check in with the monitored user, or the household or residence of the pet. The method and system may also be used by people who are not pet owners, but who would also like a simple, personalized auto-generated responsiveness check in.

### BRIEF DESCRIPTION OF THE DRAWINGS

The following description, given with respect to the attached drawings/schematic, may be better understood with reference to the non-limiting examples of the drawings/flow chart, wherein:

FIGS. 1A-1B are two parts of a flowchart showing processing steps of an exemplary embodiment of a monitoring method as described herein;

FIGS. 2A-2C are exemplary database tables as may be used to store configuration information for monitored users and their contact protocols in a method and system as described herein;

FIGS. 3A-3C are tabular representations of data structures (such as queues) at a first time storing information about when check-in messages are to be sent, when check-in messages are expected to be returned before, and what responses to check-in messages are considered overdue;

FIGS. 4A-4C are tabular representations of the queues of FIGS. 3A-3C at a second time, after the time of FIGS. 3A-3C, storing information about when check-in messages are to be sent, when check-in messages are expected to be returned before, and what responses to check-in messages are considered overdue;

FIGS. 5A-5C are tabular representations of the queues of FIGS. 4A-4C at a third time, after the time of FIGS. 4A-4C, storing information about when check-in messages are to be sent, when check-in messages are expected to be returned before, and what responses to check-in messages are considered overdue;

FIGS. 6A-6C are tabular representations of the queues of FIGS. 5A-5C at a fourth time, after the time of FIGS. 5A-5C, storing information about when check-in messages are to be sent, when check-in messages are expected to be returned before, and what responses to check-in messages are considered overdue;

FIG. 6D is a set of exemplary responses used to illustrate the kinds of messages that can be received in response to a check-in message that can be mapped back to outstanding check-in requests;

FIG. 7 is a block diagram of internal components of a system for implementing methods described herein;

FIG. 8 is an architectural diagram showing the system of FIG. 7 communicating with monitored users and ICE Contacts using a number of communications protocols (e.g., cellular, PSTN, WiFi, and Internet communications);

FIG. 9 is an exemplary interface in an app for requesting a response to a check-in message (either internally generated or in response to an externally received prompt);

FIG. 10 is an exemplary interface in an app for reminding a user that the system is still awaiting a response to a check-in message;

FIGS. 11 and 12 are exemplary interfaces in an app for obtaining additional cognitive information from the monitored user.

### DETAILED DESCRIPTION OF EXEMPLARY EMBODIMENTS

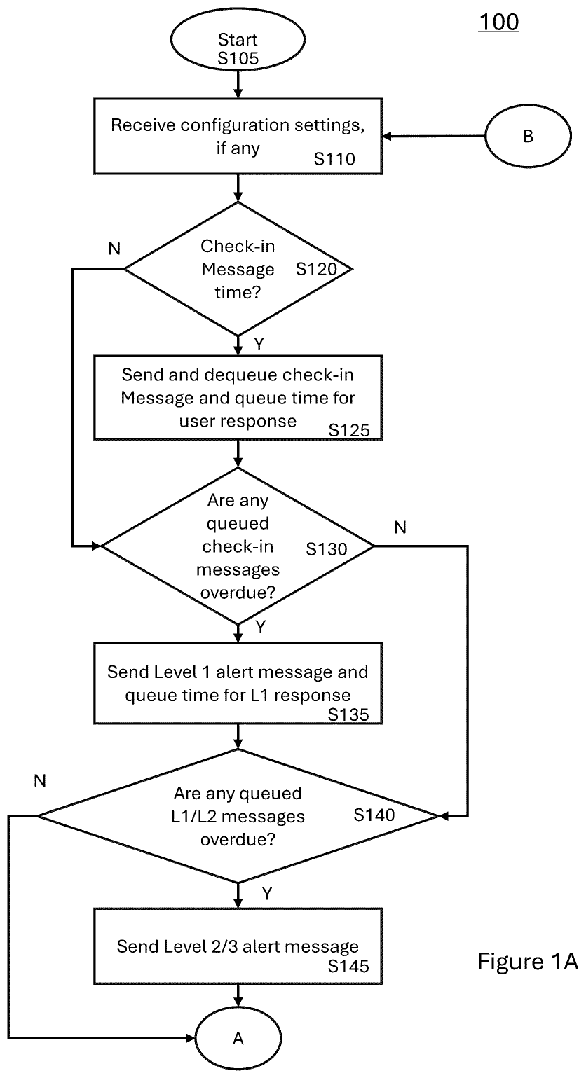

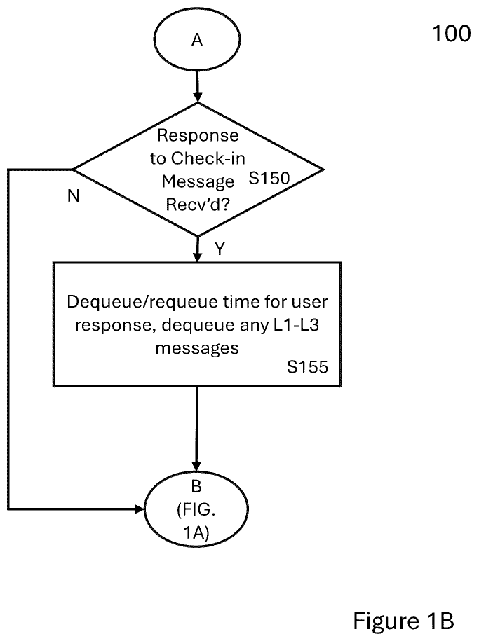

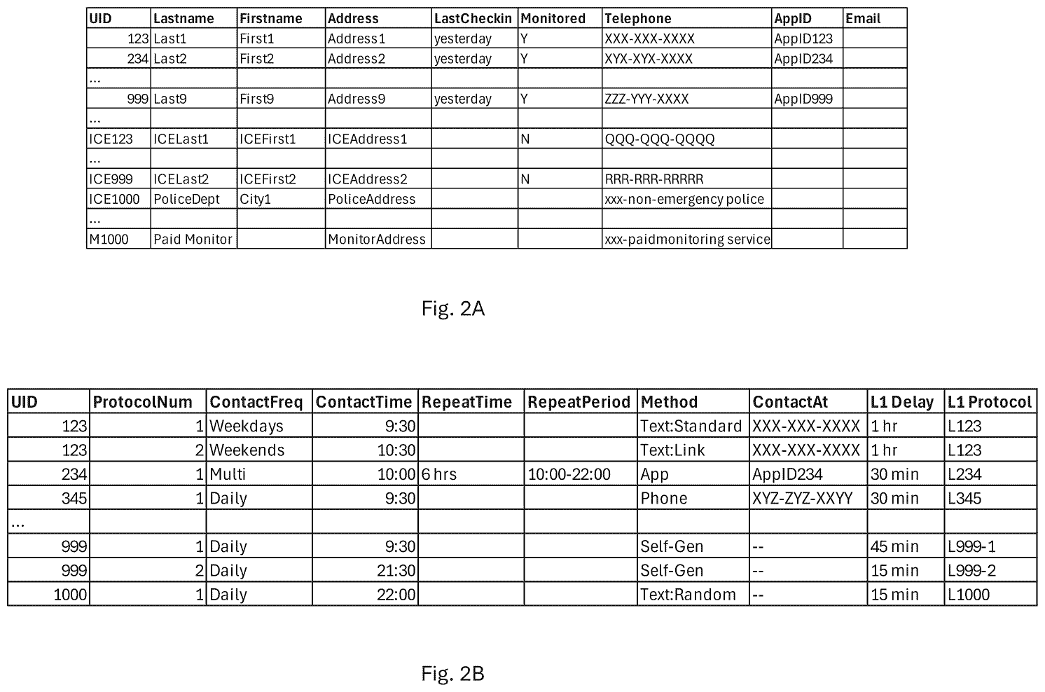

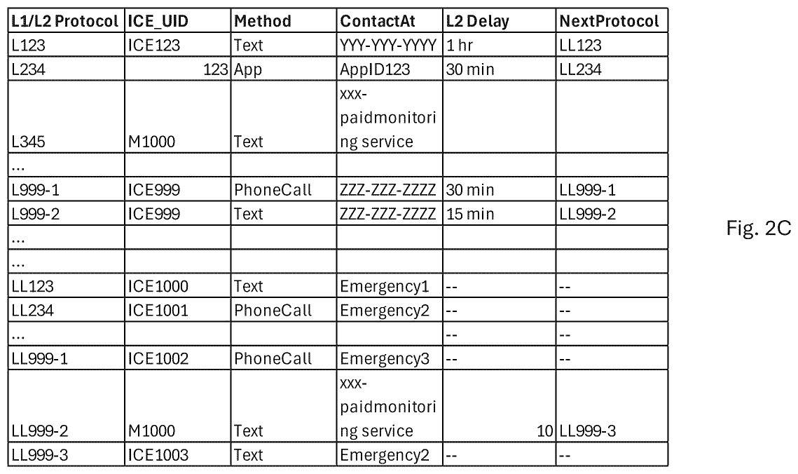

FIG. 1 is a flowchart showing processing steps of an exemplary monitoring method 100. The method starts in step S105 with any initialization required to begin the computer process that implements the method (e.g., initializing communications interfaces and reading existing configuration profiles from non-volatile storage (e.g., a database residing on a magnetic or optical medium or in a flash memory device). Configuration profiles may be included in one or more database tables such as the tables illustrated in FIGS. 2A-2C. In FIG. 2A, an exemplary set of fictitious users to be monitored are shown along with a unique identifier (UID) for each monitored user. For example, a first fictitious user named “First1 Last1” is assigned a UID=123 and generally will be referred to herein as User123. The use of a UID allows the system to commonly refer to the user in a number of inter-related tables, and, in one embodiment, the UID is a primary key or part of a primary key for many of the different tables within the system. This facilitates querying for records associated with the user by using the UID.

As shown in FIG. 2A, also associated with the user may be other information tracked by the system. Such information may include, but is not limited to, User123's address (Address1), when User123 last checked-in (shown as “yesterday”), what User123's telephone number is (XXX-XXX-XXXX), User123's Email and what the identifier is for User123's app so that User123 can be contacted via its app. The system also may track, for example, if User123 is toggled to Vacation Mode, and if they have Toggled Notify LPD to Off as well as their history of LPD change status.

Similar information is stored for other monitored users shown as having UIDs of 234 and 999. Stored in the same table or in a different table, the monitoring system may include the configuration information of the emergency contacts associated with each of the monitored users. Such contacts are often referred to as ICE (In Case of Emergency) contacts. In one embodiment, ICE contacts, like monitored users, are assigned a UID to enable faster or more efficient processing. For example, ICE contacts associated with UID=ICE123 and UID=ICE234 are stored in the table of FIG. 2A along with their telephone numbers. Given that the ICE contacts may be people that are not monitored, in one embodiment, the configuration table may include a field (e.g., Monitored) that indicates if the entry corresponds to a monitored user. However, in one embodiment, ICE contacts can be other monitored users (e.g., as shown in FIG. 2C with respect to User123 being an ICE contact for User234). In the illustrated embodiments of FIGS. 2A and 2C, User123 receives its own check-in notifications by text but notifications about User234 via its app.

Stored in the same table or in a different table, the monitoring system may include the configuration information of non-emergency public services (e.g., police, fire department, and/or welfare services) which may be contacted (serially, in parallel, or simultaneously) in some jurisdictions in case the ICE contact is not successful in getting the monitored user to respond, such as is shown with respect to UID=ICE1000. In jurisdictions that do not allow automated systems to contact non-emergency (or emergency) public services, the same or a different table may include information on a registered monitoring service that, in the event that an ICE contact is not successful in getting the monitored user to respond, may attempt to contact the monitored user and/or contact non-emergency public services on behalf of the monitored user.

In an exemplary embodiment, the monitoring system may further include a table (e.g., as shown in FIG. 2B) that includes configuration information for establishing if/how the monitored users are to receive check-in messages from the system. For example, the table of FIG. 2B shows that User123 is configured to receive weekday check-in messages via a text message (or SMS message) at 9:30 AM (as noted in the “ContactTime” field) using telephone number XXX-XXX-XXXX (as noted in the “ContactAt” field). The system is configured to allow User123 1 hour of delay (noted in “L1 delay” field) before contacting its corresponding ICE contact using a specified contact protocol (having an identifier listed in “L1 protocol” field). In this case, the system uses protocol L123 stored in the same table or in a different table. As illustrated, protocol L123 is one of several protocols stored in the table of FIG. 2C, described in greater detail below. The weekend check-in messages are sent an hour later than the weekday check-in messages and require User1 to click on a link (rather than send a return text), but otherwise the weekend protocol follows the same contact process. Alternatively, the weekend and weekdays processes could be completely different.

In some embodiment, a monitored user may be configured to have more than one ICE contact that is contacted either serially or in parallel after non-responsiveness of a monitored user for longer than the user's configured delay period. Thus, a system may support level 1 (L1) contacts, level 2 (L2) contacts, level 3 (L3) contacts, etc., and may support multiple protocols per monitored user at each level.

In contrast to User123, User234 is configured to receive multiple contacts during the day, starting at 10 AM and continuing until 10 PM (22:00) as noted in the “Contact Time” field and the “RepeatPeriod” field. As noted in the “RepeatTime” field, User234 is contacted every 6 hours during the RepeatPeriod. User234 is contacted using User234's app (as noted in the “Method” field) and may require an identifier for the user-specific app (e.g., AppID234 in the “ContactAt” field). Should User234 not respond to one of the check-in messages during the day, the system utilizes protocol L234 to alert one or more levels of ICE contacts (e.g., by sending an “overdue” message to the ICE contact(s) indicating that the corresponding user has not checked-in). The overdue message may be implemented using any of the techniques described herein.

In yet another embodiment, the system is configured to track that the app of User999 is configured to internally generate a check-in prompt twice a day (e.g., at 9:30 and 21:30). This means that the system does not have to send User999 a check-in message, but the system does have to configure itself to know when a response to the check-in prompt is overdue. Depending on which response is overdue, as illustrated, the system is configured to use two different L1 protocols when the response is overdue. Such a configuration may be beneficial when multiple caregivers are caring for a single monitored user. For example, due to work schedules, a first child may be in charge of an elderly parent in the morning, and a second child may be in charge of the elderly parent in the evening.

In yet another embodiment, the system is configured to use a telephone call, by either a human or an automated system, to contact User345. User345 is contacted at telephone number XYZ-ZYZ-XXYY, and if no response is received within 30 minutes (e.g., with call backs every 10 minutes), protocol L345 is used to inform the LI ICE contact for User345. In the context of a phone call-based inquiry, a response of the monitored user may be recorded and digitized (e.g., as part of a speech-to-text conversion) to process a response from the monitored user. Furthermore, additional information may be obtained from the monitored user during the call. Either the human or automated system may ask if the monitored user has eaten, taken its medicine, gone to the bathroom, showered, etc. Depending on the results, further processing may be performed (e.g., contacting an ICE contact or a medical professional).

Like the processing of FIG. 2B, the table of FIG. 2C shows how successive levels of ICE contacts are contacted and after what interval. At any point in time, the system will stop trying to contact further levels in the multi-level protocol if the monitored user responds to the missed check-in message.

As shown in FIG. 2C, protocol L123 (which is initiated after User123 fails to respond to a check-in message for more than 1 hour) configures the system to cause ICE123 to be contacted by text at telephone number YYY-YYY-YYYY. The system also is configured such that ICE1000 will be contacted an hour after ICE123 is contacted (assuming that User123 does not respond in the meantime). In one such configuration, an ICE contact is allowed to clear a missed check-in message for a monitored user (if agreed to by the monitored user during configuration) (e.g., as may be useful if the monitored user's phone battery is dead). In another embodiment, only the monitored user (or medical services) may clear a missed check-in message.

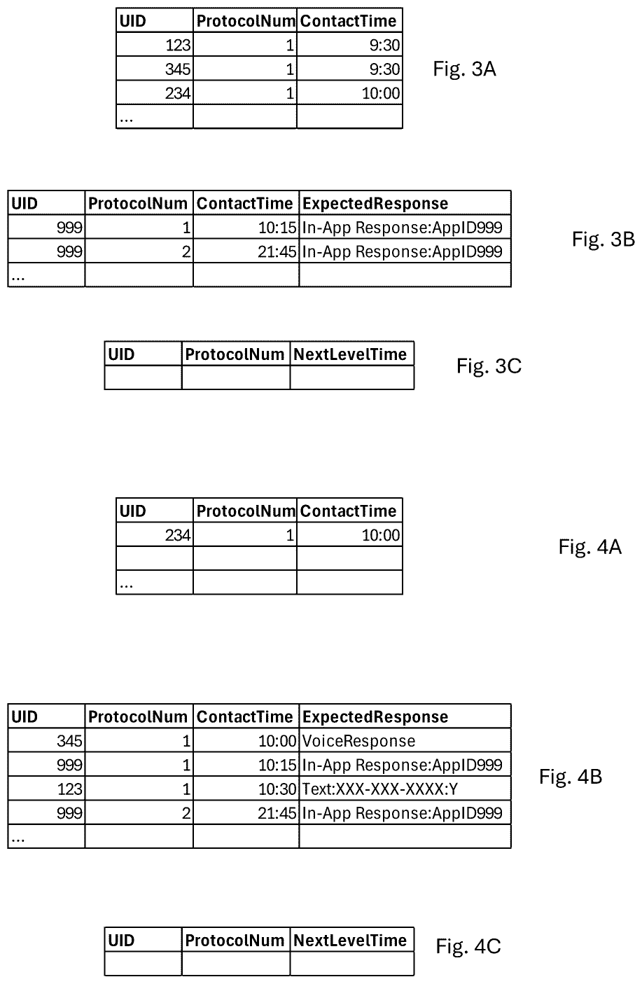

As part of the start-up process of S105, the system builds internal data structures (e.g., queues, ordered queues, lists, or ordered lists) of entries that track the times that messages need to be sent and the times that responses are considered overdue. For example, although shown in table form in FIG. 3A, the monitoring system builds (e.g. by processing the configuration information of FIG. 2B) a queue representing the check-in messages that need to be sent and the times at which the check-in messages need to be sent. As shown, the table of FIG. 3A represents the “check-in messages to be sent” queue as it stands at 9:29 AM on a particular day. As shown, the “check-in messages to be sent” queue has been time ordered for faster processing and does not include entries for User999 because those messages are internally generated within User999′s app (and therefore do not need to be sent). The generation of the information in the queue may be generated once and then continuously updated, or the information in the queue may be generated or updated daily (or multiple times daily, such as every hour) so that the number of entries tracked by the system at any one time is reduced (e.g., by clearing entries that were responded to one day that do not need to be readded to the queue(s) until the next day).

The system would also generate an initially empty queue of “awaiting a response” entries assuming that the queue did not yet exist. The system, however, would then add to the “awaiting a response” queue two entries for User999 corresponding to when User999's responses should be expected to be received by. As shown, those entries correspond to expected response by 10:15 and 21:45, respectively. The resulting queue illustrated in table form is shown in FIG. 3B.

As there are no overdue messages, the system creates an empty “overdue messages” queue, as shown in FIG. 3C, if the queue does not already exist. After creation of the empty queue, in the illustrated exemplary embodiment, step S105 finishes. As noted above, the configuration parameters may be reloaded daily (e.g., at midnight) to reduce memory usage and speed processing time of adding to, removing from, and reading from the queues.

After the processing in step S105, control then passes to step S110 where the method receives (e.g., directly from a user by way of a mobile “app” communicating with the computer process implementing the method or by reading from a database a new entry added to the database on behalf of a user that, for example, registered using a WWW browser) any new configuration settings, if any, for any new users or users with modified settings. The configuration setting may include, but are not limited to, the configuration settings of any one of FIGS. 2A-2C. Any check-in messages that need to be queued due to the new data are added to the corresponding queue. Likewise, any “awaiting a response” entries that need to be added to the queue are added.

Control then passes to step S120 where the process of processing the queues begins (or continues). In step S120, the system checks the “check-in messages to be sent” queue to see if there are any check-in messages to be sent. Assuming that the time has become 9:30, the system detects that there are check-in messages to be sent for User123 and User345. Control then passes to Step S125, and the system performs the check-in messaging according to (UserID=123, ProtocolNum=1) and (UserID=123, ProtocolNum=1) by finding their corresponding entries in the table of FIG. 2B. The corresponding entries can then be removed (i.e., dequeued) from the “check-in messages to be sent” queue. The resulting queue is shown in FIG. 4A.

In addition, the system queues response times in the “awaiting a response” queue. The resulting “awaiting a response” queue, if implemented as an ordered queue, is illustrated in FIG. 4B. Control then passes from S125 to S130. As of 9:30, there are no overdue check-in messages (as shown in FIG. 4B), so control passes to step S140.

In step S140, the system determines if there are any queued overdue L1/L2 messages (as shown in FIG. 4C), and finds that there are none, so control passes to step S150 on FIG. 1B. As would be appreciated by those of skill in the art, an arbitrary number of levels can be tracked for each monitored user and stored in the same or a different queue. For example, as shown in FIG. 2C, the L999-2 protocol of User999 begins a three level protocol (e.g., L999-2, LL999-2, and LL999-3) using texts to an ICE contact, a monitoring service, and emergency services, respectively, after 15 minute and 10 minute delays at the first two levels.

In step S150, the system determines if there are any response messages that have been received from any monitored users. If there are, the corresponding entries from the queues can be removed. Assuming that, as of 9:31 AM there are no received response messages, control passes back to Step S110 of FIG. 1A, and the queues of FIGS. 4B and 4C remain unchanged.

The process of FIGS. 1A and 1B continues periodically (e.g., every 30 second, 1 minute 5 minutes, or until whatever is the earliest time to perform the next activity in any of the queues). For example, as the earliest next time for processing is 10:00 in any of FIGS. 4A-4C, the system could sleep until then or until an incoming message is received.

The processing steps of FIGS. 1A and 1B are shown as being performed sequentially; however, the method may be performed in parallel (e.g., using multiple threads of a processor or multiple processes on one or more processors) as well. In such a parallel approach, locking is performed on any of the shared data structures and/or message passing is performed between threads and/or processes to ensure that multiple threads or processes are not making changes to the same data simultaneously.

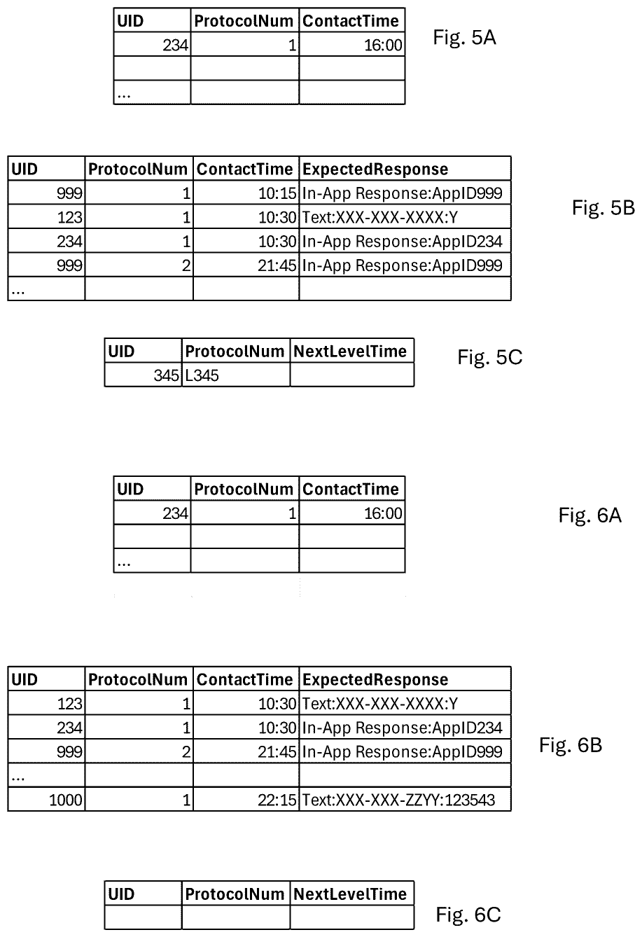

Assuming that the time has become 10:00, the next time that the steps S110-S155 are performed, the queues again will be processed. In step S120, the queue of FIG. 4A will be checked, and the system will determine that the contact time for UID 234 (ProtocolNum=1) has arrived. Control therefore passes to step S125, and the check-in message is sent to the User234 via User234's app (with an ID of “AppID”), and the need to respond to that message is queued in the “awaiting a response” queue. Also, since UID 234 (ProtocolNum=1) provides multiple messages daily, its next message is queued in the “check-in messages to be sent” queue (as shown in FIG. 5A).

Control then passes to step S130, and the system determines whether there are any check-in messages that are overdue. The check-in message for User345 is found to be overdue, so control passes to step S135. In step S135, the system automatically/programmatically determines that User345 has a response that is overdue. The system automatically/programmatically then activates protocol L345. The system does so by looking up L345 in the database, determining that M1000 should be contacted by text at phone number “xxx-paidmonitoring service,” and sends the text to the corresponding number. The system also automatically/programmatically determines that there are no subsequent follow-up actions to be taken after that and queues the resulting entry in the “overdue messages” queue. The resulting queues are shown in FIGS. 5A-5C.

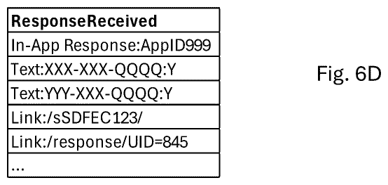

Assuming that User999 and User345 respond before the next time that step S150 is performed (e.g., at 10:05), the resulting queues before the processing at 10:15 are shown in FIGS. 6A-6C.

The check-in messages can be acknowledged in a number of ways. In a first embodiment, the monitored user receives a check-in message, either by a text message (via a cellular network) or via a TCP/IP-based communication (such as a direct communication with the user's app or an iMessage) (as either a push-notification or via polling at least one server for new messages). Having received the check-in message, a user, if their phone is locked, may nonetheless see that there is a notification (e.g., a text notification, an iMessage notification, or an app notification) that requires the phone to be unlocked. When the user unlocks the phone, the receiving interface displays the message. For example, the text message or iMessage interface displays a message from the system that the user has to interact with or respond to. For example, in the case of a text message, the user may have to respond with a fixed, repeatable response (e.g., “Ok” or “y”) to indicate that the user is well, or a different fixed response (e.g., ‘N’) if the user is not well. The fixed, repeatable responses can be listed in the message (“Are you okay. Reply ‘Y’ for yes and ‘N’ for no.”). In an alternate embodiment (e.g., when a user is configured to receive check-in messages with the “Text: Random” Method), the system includes in the sent message a more complicated, time varying response request (e.g., enter the displayed 6-digit number) to require an increased level of engagement with the system. Alternatively, the user may be requested to respond with a secret code.

In yet another embodiment (e.g., when a user is configured to receive check-in messages with the “Text:Link” Method), the message may include a link to a website that the user must select such that the browser of the user's mobile device is activated. In one embodiment, the system tracks that the link has been requested and uses that as confirmation that the user is well. In an alternate embodiment, the user is obligated to interact with the website corresponding to the link to provide confirmation feedback. However, since the use of a browser may require a data connection (as opposed to a cellular connection), a user may configure how it wishes to receive check-in messages (as described above with respect to FIGS. 2B and 2C).

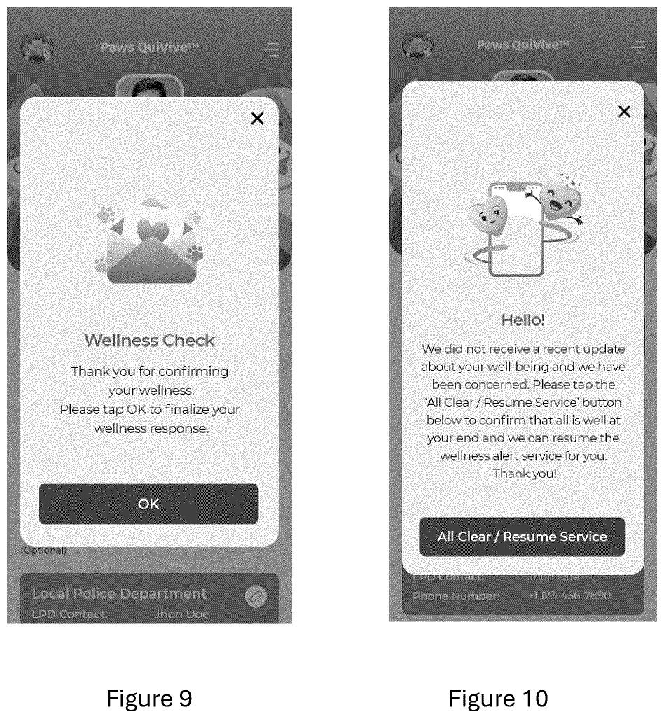

In yet another embodiment, the user receives check-in messages via an app specific to wellness checks (e.g., using an interface such as is shown in FIG. 9), and the app may control various aspects of how the user responds to the check-in messages. For example, as shown, the user need only click on the “OK” button. Alternatively, the app may accept voice responses in addition to or instead of touch screen interfaces. The app also may ask the monitored user to record a message to an ICE contact should the ICE contact wish to hear that the monitored user is okay. Using an app, the system has greater control over attempting to get the attention of a monitored user when the monitored user has not responded. For example, the sound level of the alert, the frequency of the alert, whether the phone/tablet flashes or vibrates may be controlled so the monitored user is more likely to realize that it is missing a check-in message.

As shown in FIG. 10, the app also may change the interface of FIG. 9 to remind the monitored user that the system is still waiting for a response.

In one embodiment, the method performs an acceptance check to ensure that the level 1 and/or level 2 ICE contacts have agreed to be contacts for the registering user. This may include sending a message to the contact using the registered method and/or receiving confirmation from the registering user to be monitored that the contacts have indicated their agreement. In the case of a level 1 or level 2 contact not having agreed or withdrawing consent to be a contact, the monitored user will receive a message indicating that a new contact is needed, and the information on a new contact can be received (e.g., using an in-app interface, using a response to a text message, and/or by providing the details by phone or via a WWW browser).

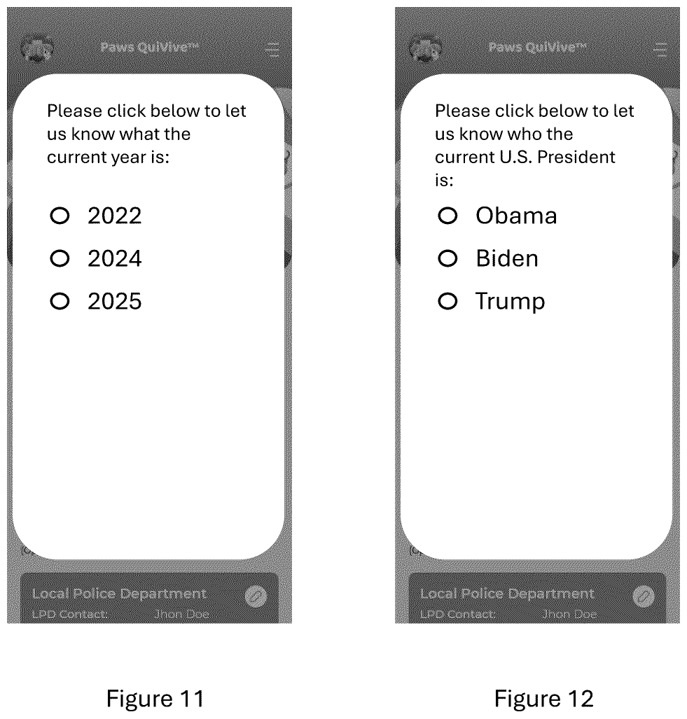

In an alternate embodiment, step S110 further includes receiving additional information that allows checking beyond mere responsiveness to a received prompt/message. In such an embodiment, the method receives as part of the configuration information one or more configurable cognitive prompts and the correct answers to those cognitive prompts if not already known or stored by the system. Cognitive prompts can be included as part of the check-in prompt to the user at the time of receipt of the check-in prompt. For example, the method may receive information such that the user may be prompted with certain user-independent information (e.g., ‘What year is it?’ or ‘Who is the current president of the United States’) as shown in FIGS. 11 and 12, or certain user-specific information (e.g., ‘In what year were you born?’ or ‘Which of the following names is your mother's name?’). The method may further be configured with a standard or user-specific time period in which, once prompted for the answer, the user must correctly respond to the question(s) of the cognitive prompt. The question(s) may be the same or different each day. In one such embodiment, the app on the mobile device (or the remote monitoring method) records how long it takes for the user to answer the question(s) of the cognitive prompts, and if the user takes longer than average (e.g., 50%, 100%, or 150% longer) to answer the cognitive prompts or answers the cognitive prompts incorrectly, then the level 1 contact may be informed using a “cognitive concern” message what cognitive issue has arisen (e.g., that the user took too long to answer or answered incorrectly). In some embodiments, some of the cognitive prompts may be shared between multiple users and the system just selects one or more of the shared cognitive prompts at random to display to the user. The amount of time to respond to the cognitive prompts may be configurable on a per-user basis, may be standard for all users, or may be set by some demographic information (e.g., users under 70 should answer in 5 seconds, and users over 70 but under 90 should answer in 10 seconds).

In an alternate embodiment, the user's time period for responding may be altered by other factors that can be configured for the user. For example, if a user's premises are equipped with a motion detector linked to the system (or the user's mobile device), and the monitored user is freely moving around in the home (as opposed to being stuck on the floor or in the bathroom), the system may either provide additional time for a response (assuming that the user is just away from its mobile device) and/or send the additional information about movement to the next level contact.

In a further alternate embodiment, in addition to being provided a prompt by the user's mobile device when it is time to respond, the user's mobile device may, during or after the prompt, display to the user advertising or other promotional material as well as reminders specific to the user (e.g., a reminder to (1) feed the pet(s), (2) buy a particular product, or (3) take a particular medication). In the case of a reminder to take a medicine, the configurable times at which the user is to be prompted can be configured to coincide with the one or more time periods during the day at which the monitored user should take his/her medicine.

In yet another embodiment, the monitored user may indicate to the monitoring method and system (via a wellness app, the web, or a DTMF interface) that monitoring is to be temporarily suspended (e.g., while the user is away on vacation) or is to be time shifted (e.g., by changing all times by an appropriate number of hours when staying in a different time zone).

In addition to being able to respond to various check-in messages, in one embodiment, a monitored user may utilize its wellness app to update the configuration parameters stored within the system to affect changes in how the system interacts with the monitored user. Alternatively, a care giver of ICE contact may be provided with an interface (e.g., via the WWW or in an app) to change the information on behalf of the user.

The above discussion is not limited to configurations in which the monitored user utilizes an app (e.g., on a smartphone or tablet). Instead, the monitored user may register the monitored user (or an ICE contact may register the monitored user with the monitored user's consent), either using an app or a web interface, and the monitored user may then confirm acceptance of the monitoring and/or respond to check-in requests using a conventional PSTN telephone or basic cellular phone without smartphone capabilities. In such configurations, the monitored user receives check-in requests by voice or by standard text message without the need for a smartphone or tablet.

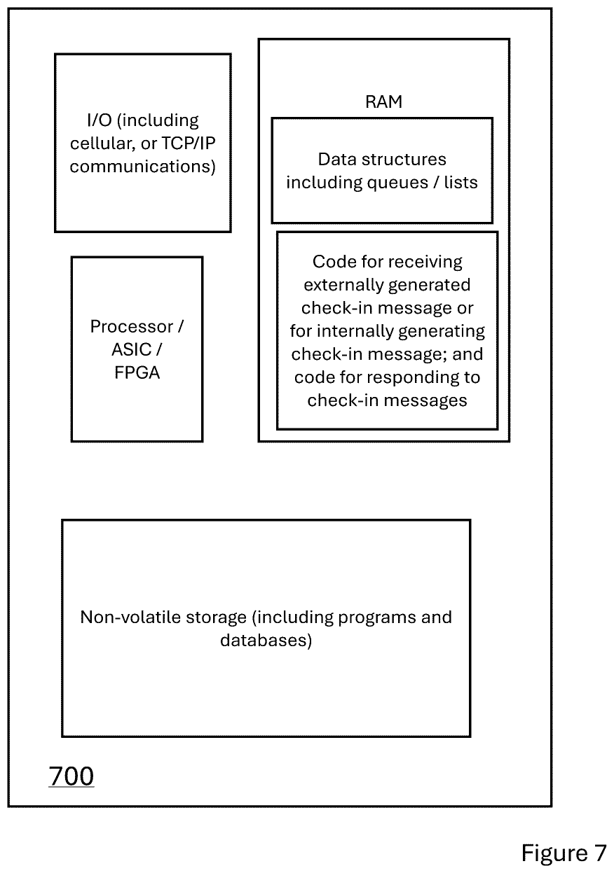

The monitoring system and method may be implemented as a processing circuitry-based system and method. For example, a system 700 (FIG. 7) for performing the method may include, but is not limited to, a memory such as a recording medium including a read-only memory (ROM) and a random access memory (RAM) in addition to an external memory device such as a hard disk drive (HDD) and an optical disc device. The memory stores various programs executed by a processor of the processing circuitry as well as various types of data and information.

An input interface for the system includes various devices for an operator to input various types of information and data, and is configured to include a mouse, a keyboard, a trackball, and/or a touch panel, for example.

The processing circuitry is a circuit equipped with a central processing unit (CPU) and/or a special-purpose or general-purpose processor, for example. The processor implements various functions described above by executing the programs stored in the memory. The processing circuitry may be configured as hardware such as an FPGA and an ASIC. The various functions described above can also be implemented by such hardware. Additionally, the processing circuitry can implement the various functions by combining hardware processing and software processing based on its processor and programs.

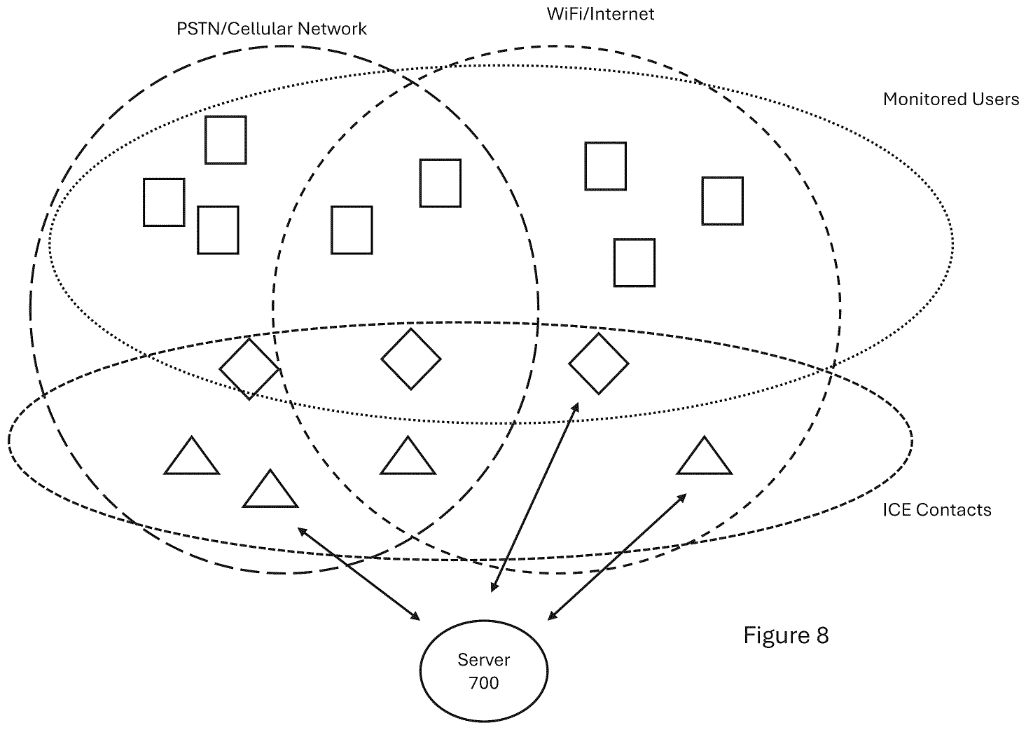

FIG. 8 is an architectural diagram showing the system of FIG. 7 communicating with monitored users and ICE Contacts using a number of communications protocols (e.g., cellular, PSTN, WiFi, and Internet communications). As illustrated, and as described herein, monitored users also may be ICE contacts for other users.

The methods described herein can be performed using interpreted code, compiled code, or a combination of both. The code being executed is stored in a computer readable medium, and, in general, a computer program product includes a computer storage medium and computer instructions configured to cause at least one computer processor to execute computer instructions for causing the at least one computer processor to performs the steps described herein.

This application contains the description of implementations and embodiments with the help of examples. It will be appreciated by a person skilled in the art that the present invention is not restricted to details of the embodiments presented above, and that the invention can also be implemented in another form without deviating from the scope of the appending claims. The embodiments presented above should be considered illustrative, but not restricting. Consequently, various options of implementing the invention as determined by the claims, including equivalent implementations, also belong to the scope of the invention.

1. A method of remotely monitoring the health of a person, comprising:
determining when a response to a check-in message of a first monitored user is overdue; and
automatically sending an “overdue” message to a first registered “in case of emergency” (ICE) contact of the first monitored user to indicate that the response to the check-in message of the first monitored user is overdue.

2. The method as claimed in claim 1, further comprising sending the check-in message to the first monitored user as a text message.

3. The method as claimed in claim 1, further comprising sending the check-in message to the first monitored user as a short message service (SMS) message.

4. The method as claimed in claim 1, wherein the check-in response is internally generated by an app of the first monitored user.

5. The method as claimed in claim 1, further comprising sending the check-in message to the first monitored user as a TCP/IP message.

6. The method as claimed in claim 5, wherein the TCP/IP message is an iMessage.

7. The method as claimed in claim 1, further comprising sending the check-in message to the first monitored user as a phone call.

8. The method as claimed in claim 7, wherein the phone call is a phone call from a human.

9. The method as claimed in claim 7, wherein the phone call is a phone call from an automated dialing system for recording a response from the first monitored user.

10. The method as claimed in claim 1, further comprising clearing that the response to the check-in message is overdue after receiving a text response to the check-in message.

11. The method as claimed in claim 1, further comprising clearing that the response to the check-in message is overdue after receiving a short message service (SMS) message in response to the check-in message.

12. The method as claimed in claim 1, further comprising clearing that the response to the check-in message is overdue after receiving an indication that a link included in the check-in message has been accessed.

13. The method as claimed in claim 1, further comprising receiving cognitive check information as part of the response to the check-in message of the first monitored user.

14. The method as claimed in claim 1, further comprising clearing that the response to the check-in message is overdue after receiving an indication that an in-app user interface control has been accessed.

15. The method as claimed in claim 13, further comprising clearing a cognitive check when the cognitive check information received as part of the response to the check-in message of the first monitored user is correct.

16. The method as claimed in claim 13, further comprising clearing a cognitive check when the cognitive check information received as part of the response to the check-in message of the first monitored user is correct and was answered within a threshold period of time.

17. The method as claimed in claim 13, further comprising automatically sending a cognitive issue message to the first ICE contact of the first monitored user when the cognitive check information received as part of the response to the check-in message of the first monitored user is at least one of (a) incorrect and (b) not answered within a threshold period of time.

18. The method as claimed in claim 1, wherein automatically sending the message to the first registered ICE contact of the first monitored user to indicate that the response to the check-in message of the first monitored user is overdue comprises automatically sending the message to the first registered ICE contact of the first monitored user and to a second registered ICE contact of the first monitored user to indicate that the response to the check-in message of the first monitored user is overdue.

19. The method as claimed in claim 1, wherein automatically sending the message to the first registered ICE contact of the first monitored user to indicate that the response to the check-in message of the first monitored user is overdue comprises automatically sending the message to the first registered ICE contact of the first monitored user and to a second registered ICE contact of the first monitored user simultaneously to indicate that the response to the check-in message of the first monitored user is overdue.

20. A system for remotely monitoring the health of a person, comprising:
a processor; and
a non-transitory computer readable medium for storing instructions, which, when executed by the processor, cause the processor to:
determine when a response to a check-in message of a first monitored user is overdue; and
automatically send a message to a first registered “in case of emergency” (ICE) contact of the first monitored user to indicate that the response to the check-in message of the first monitored user is overdue.
